(19) United States
(12) Patent Application Publication
(10) Pub. No.: US 2007/0270639 A1
(43) Pub. Date: Nov. 22, 2007

(54) Medical instrument having a catheter and having a catheter accessory device and method for using

(76) Inventor: Gary L. Long, Cincinnati, OH (US)

Correspondence Address: THOMPSON HINE L.L.P.;Intellectual Property Group, P.O. BOX 8801, DAYTON, OH, 45401-8801, US

(21) Appl. No.: 11/435,551

(22) Filed: May 17, 2006

(51) Int. Cl. A61B 1/00

(52) U.S. Cl. 600/106; 600/104; 600/129

(57) ABSTRACT

A medical instrument includes a flexible catheter and a catheter accessory device. The catheter has a distal end insertable within a body lumen of a patient. The device is slidably engagable with the catheter. One of the catheter and the device includes magnetic material, and the other of the catheter and the device includes electrically-energizable windings. The catheter and the device are adapted to operate as a linear motor wherein controllably electrically energizing the windings while immobilizing one of the catheter and the device slidingly moves the other of the catheter and the device. A method for using the medical instrument, wherein the medical instrument also includes a controller operatively connected to the windings, includes inserting the distal end within the body lumen, immobilizing one of the catheter and the device, and activating the controller to advance the other of the catheter and the device within the body lumen.

### FIELD OF THE INVENTION

The present invention is related generally to medical equipment, and more particularly to a medical instrument having a catheter and having a catheter accessory device and to a method for using the medical instrument.

### BACKGROUND OF THE INVENTION

Examples of known catheters include, without limitation, flexible insertion tubes of endoscopes (including flexible insertion tubes of colonoscopes and enteroscopes). The insertion tube has an articulatable distal end portion controlled by wires running from the distal end portion to control knobs on the handle of the endoscope. A wide angle video camera in the distal end of the insertion tube permits medical observation. In use, the distal end of the insertion tube is inserted into a body lumen of a patient. The user manually pushes on a proximal tube portion to advance the distal end of the insertion tube within the body lumen for medical observation and/or medical treatment. In a serpentine body lumen, such as the colon, the articulatable distal end of the insertion tube can become misaligned in the body lumen and become blocked by lumen tissue from further advancement. Then, if the user further pushes on the proximal tube portion, the insertion tube forms undesirable loops which the user must correct before realigning the distal end of the insertion tube and further advancing the insertion tube within the body lumen.

Typically, an accessory medical device is part of an endoscopic system. In one example, the accessory device is an end effector (such as a medical snare, a medical grasper, etc.) which is inserted into a working channel of the insertion tube of the endoscope and is manually translated to extend from the distal end of the working channel for medical observation and/or medical treatment. In another example, the accessory device is a flexible accessory tube adapted to be coupled to, and manually translated along, an exterior rail of the insertion tube of an endoscope after the insertion tube has been advanced to a target site within the body lumen. Then, an end effector is inserted into an accessory working channel of the accessory tube and is manually translated to extend from the distal end of the accessory working channel for medical observation and/or medical treatment. Pushing on the proximal end of the accessory tube to translate the accessory tube along the exterior rail can also lead to the formation of undesirable loops.

It is known to manually advance a guidewire within a body lumen and then to manually advance a catheter along the guidewire.

Still, scientists and engineers continue to seek improved medical instruments having a catheter and having a catheter accessory device and improved methods for using such medical instruments.

### SUMMARY OF THE INVENTION

A first expression of an embodiment of the invention is for a medical instrument including a flexible medical catheter and a catheter accessory device. The catheter has a distal end insertable within a body lumen of a patient. The device is slidably engagable with the catheter. One of the catheter and the device includes magnetic material, and the other of the catheter and the device includes electrically-energizable windings. The catheter and the device are adapted to operate as a linear motor wherein controllably electrically energizing the windings while immobilizing one of the catheter and the device slidingly moves the other of the catheter and the device.

A second expression of an embodiment of the invention is for a medical instrument including a flexible medical catheter, a catheter accessory device, and a linear motor controller. The catheter has a distal end insertable within a body lumen of a patient. The device slidably engages the catheter. One of the catheter and the device includes magnetic material, and the other of the catheter and the device includes electrically-energizable windings. The catheter and the device are adapted to operate as a linear motor wherein controllably electrically energizing the windings while immobilizing one of the catheter and the device slidingly moves the other of the catheter and the device. The controller is operatively connected to the windings.

A method of the invention is for using a medical instrument. The medical instrument includes a flexible medical catheter, a catheter accessory device, and a linear motor controller. The catheter has a distal end insertable within a body lumen of a patient. The device slidably engages the catheter. One of the catheter and the device includes magnetic material, and the other of the catheter and the device includes electrically-energizable windings. The catheter and the device are adapted to operate as a linear motor wherein controllably electrically energizing the windings while immobilizing one of the catheter and the device slidingly moves the other of the catheter and the device. The controller is operatively connected to the windings. The method includes inserting the distal end within the body lumen. The method also includes immobilizing one of the catheter and the device. The method also includes activating the controller to advance the other of the catheter and the device within the body lumen.

Several benefits and advantages are obtained from one or more of the expressions of an embodiment and the method of the invention. In a first example, the device is a medical guidewire which has been immobilized after having been manually advanced within the body lumen to extend from the catheter wherein controllably electrically energizing the windings slidingly moves the catheter along the guidewire. In one illustration of this example, the force to advance the catheter is applied proximate the distal end of the catheter so that the self-advancing catheter will not form undesirable loops, as can be appreciated by those skilled in the art. In a second example, the device includes a flexible accessory tube, and the catheter is immobilized after having been manually advanced within the body lumen wherein controllably electrically energizing the windings slidingly moves the accessory tube along an exterior rail of the catheter. In one illustration of this example, the force to advance the accessory tube is applied proximate the distal end of the accessory tube so that the self-advancing accessory tube will not form undesirable loops, as can be appreciated by those skilled in the art. In one employment of either or both examples, the self-advancing catheter and/or the self-advancing accessory tube has a greater advancement speed, than that obtained with manual advancement, which reduces the time required for a medical procedure.

### BRIEF DESCRIPTION OF THE FIGURES

FIG. 1 is a schematic view of an embodiment of a medical instrument including a medical catheter, a catheter accessory device in the form of a catheter-guiding member which is a catheter guide wire, and a controller, wherein the catheter includes windings, wherein the guidewire includes magnetic material, and wherein the controller is located in a handpiece which is shown in cut-away;

FIG. 2 is a cross sectional view of the distal portion of the medical catheter of FIG. 1 inserted within a body lumen of a patient, wherein the medical catheter of FIG. 2 has been rotated ninety degrees from the orientation shown in FIG. 1;

FIG. 3 is a schematic wiring diagram showing the controller electrically connected to the windings;

FIG. 4 is a perspective view of a distal portion of a first alternate embodiment of the medical instrument of FIG. 1, wherein the catheter-guiding member is a catheter guide ribbon;

FIG. 5 is side elevational view of a distal portion of a second alternate embodiment of the medical instrument of FIG. 1, wherein the catheter includes an exterior rail and wherein the catheter accessory device is adapted to slide along the exterior rail and includes a flexible accessory tube having an accessory working channel;

FIG. 6 is a view taken along lines 6-6 of FIG. 5; and

FIG. 7 is a cross sectional view of the catheter accessory device of FIG. 6 taken along lines 7-7 of FIG. 6.

### DETAILED DESCRIPTION OF THE INVENTION

Before explaining the present invention in detail, it should be noted that the invention is not limited in its application or use to the details of construction and arrangement of parts illustrated in the accompanying drawings and description. The illustrative embodiments of the invention may be implemented or incorporated in other embodiments, variations and modifications, and may be practiced or carried out in various ways. Furthermore, unless otherwise indicated, the terms and expressions employed herein have been chosen for the purpose of describing the illustrative embodiments of the present invention for the convenience of the reader and are not for the purpose of limiting the invention.

It is understood that any one or more of the following-described expressions, embodiments, examples, etc. can be combined with any one or more of the other following-described expressions, embodiments, examples, etc.

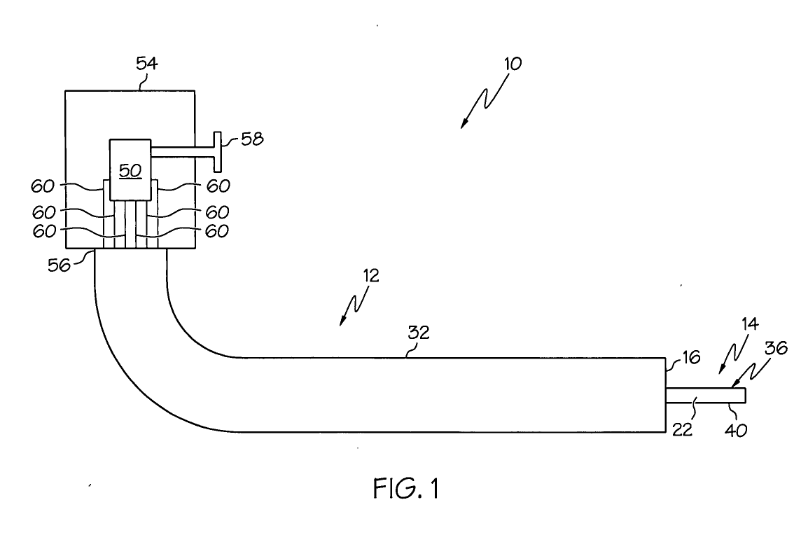

Referring now to the Figures, wherein like numerals represent like elements throughout, FIGS. 1-3 illustrate an embodiment of the invention. A first expression of the embodiment of FIGS. 1-3 is for a medical instrument 10 including a flexible medical catheter 12 and a catheter accessory device 14. The catheter 12 has a distal end 16 insertable within a body lumen 18 of a patient 20. The device 14 is slidably engagable with the catheter 12. One of the catheter 12 and the device 14 includes magnetic material 22, and the other of the catheter 12 and the device 14 includes electrically-energizable windings 24, 26 and 28. The catheter 12 and the device 14 are adapted to operate as a linear motor 30 wherein controllably electrically energizing the windings 24, 26 and 28 while immobilizing one of the catheter 12 and the device 14 slidingly moves the other of the catheter 12 and the device 14.

It is noted that the terminology “magnetic material” includes material capable of being magnetized and includes permanent magnets. It is also noted that various designs and the operation of a linear motor are well known in the art. The number of turns in a winding and the number and arrangement of the windings are left to the artisan.

In one application of the first expression of the embodiment of FIGS. 1-3, the catheter 12 is a flexible endoscope insertion tube 32. In one variation, the flexible endoscope insertion tube 32 has a single working channel 34. Non-endoscope applications are left to those skilled in the art.

In a first implementation of the first expression of the embodiment of FIGS. 1-3, the device 14 is a flexible catheter-guiding member 36. In one variation, the catheter 12 includes a catheter lumen 38, and the member 36 is disposable in the catheter lumen 38. In one modification, the catheter 12 includes the windings 24, 26 and 28, and the windings 24, 26 and 28 are disposed proximate the distal end 16 of the catheter 12. In a first example, the member 36 is a catheter guide wire 40. In one choice of materials, the guide wire 40 comprises iron.

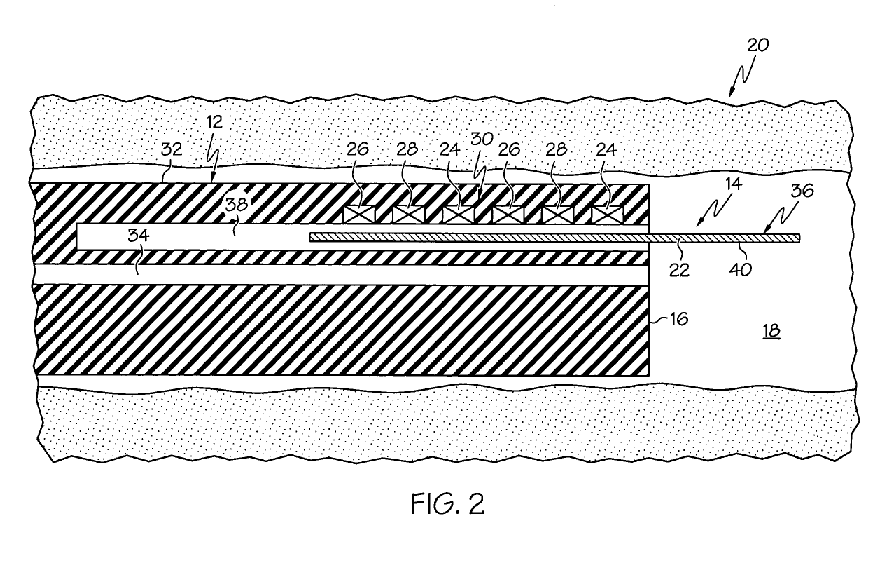

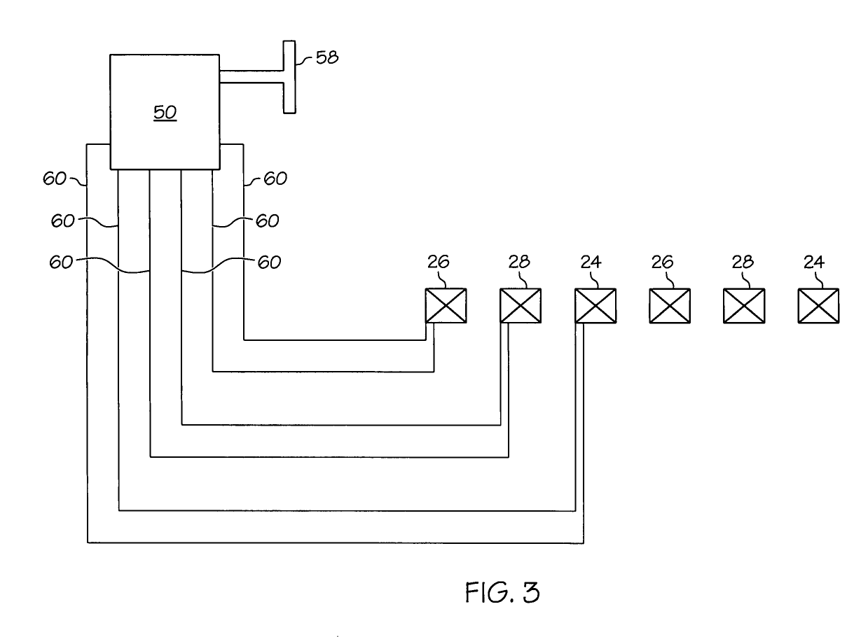

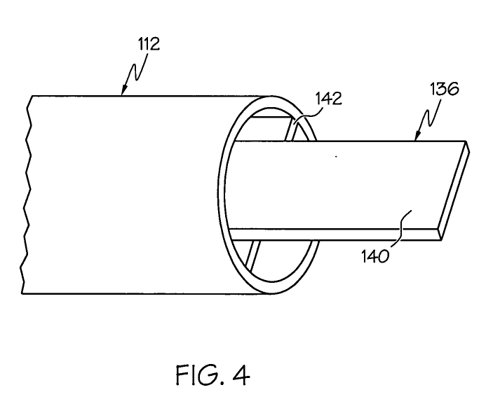

In a second example, shown in the first alternate embodiment of FIG. 4, the member 136 is a catheter guide ribbon 140 which provides a larger area allowing for a larger magnetomotive force as can be appreciated by the artisan. In one variation, the guide ribbon 140 slidingly engages a lumen-dividing wall 142 within the catheter 112. In one modification, the wall 142 includes the windings (not shown in FIG. 3, but a cross sectional view of FIG. 3 showing the windings would look similar to the view of FIG. 2). In one choice of materials, the guide ribbon 140 comprises iron.

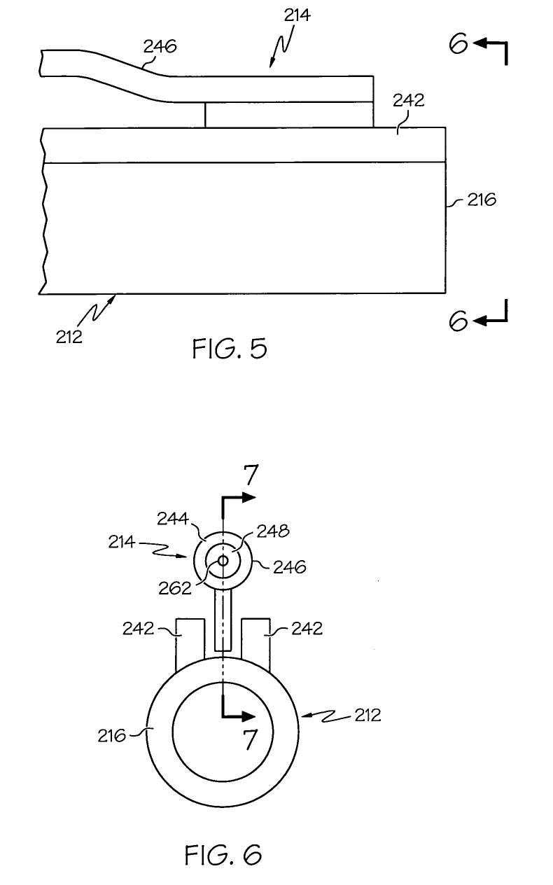

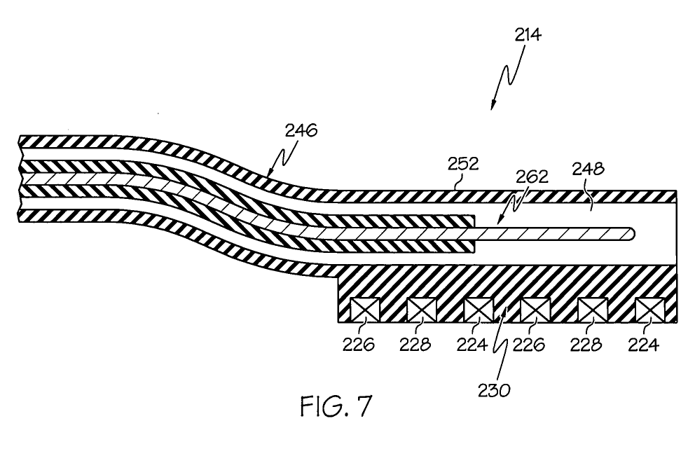

In a second implementation, as shown in the second alternate embodiment of FIGS. 5-7, the catheter 212 includes an exterior rail 242, and the device 214 is adapted to slide along the exterior rail 242 during operation of the linear motor 230. In one variation, the device 214 has a distal end 244, the device 214 includes the windings 224, 226 and 228, and the windings 224, 226 and 228 are disposed proximate the distal end 244 of the device 214. In one example, the device 214 includes a flexible accessory tube 246 having an accessory working channel 248. In one choice of materials, the exterior rail 242 comprises an elastomer having iron particles (omitted from FIG. 7 for clarity) embedded therein. In a first construction, the device 214 is coupled to the exterior rail 242 by magnetic force, and a stop (not shown) at the distal end 216 of the catheter 212 limits travel of the accessory tube 246. In a second construction, not shown, the device is coupled to the exterior rail by a tongue and groove arrangement. Other coupling constructions are left to the artisan.

A second expression of the embodiment of FIGS. 1-3 is for a medical instrument 10 including a flexible medical catheter 12, a catheter accessory device 14, and a linear motor controller 50. The catheter 12 has a distal end 16 insertable within a body lumen 18 of a patient 20. The device 14 slidably engages the catheter 12. One of the catheter 12 and the device 14 includes magnetic material 22, and the other of the catheter 12 and the device 14 includes electrically-energizable windings 24, 26 and 28. The catheter 12 and the device 14 are adapted to operate as a linear motor 30 wherein controllably electrically energizing the windings 24, 26 and 28 while immobilizing one of the catheter 12 and the device 14 slidingly moves the other of the catheter 12 and the device 14. The controller 50 is operatively connected to the windings 24, 26 and 28.

In one application of the second expression of the embodiment of FIGS. 1-3, the catheter 12 is a flexible endoscope insertion tube 32.

In a first implementation of the second expression of the embodiment of FIGS. 1-3, the device 14 is a flexible catheter-guiding member 36. In one variation, the catheter 12 includes a catheter lumen 38, and the member 36 is disposed in the catheter lumen 38. In one employment, the controller 50, when activated by a user, controllably electrically energizes the windings 24, 26 and 28 to slidingly move the catheter 12 along the member 36 when the member 36 is immobilized by the user.

In a second implementation, as shown in the second alternate embodiment of FIGS. 5-7, the catheter 212 includes an exterior rail 242, and the device 214 is adapted to slide along the exterior rail 242 during activation of the controller (the controller is shown as controller 50 in FIGS. 1 and 3). In one example, the device 214 includes a flexible accessory tube 246 having an accessory working channel 248 and a distal accessory tube portion 252, wherein the controller (the controller is shown as controller 50 in FIGS. 1 and 3), when activated by a user, controllably electrically energizes the windings 224, 226 and 228 to slidingly move the distal accessory tube portion 252 along the exterior rail 242 when the catheter 212 is immobilized by the user.

In one enablement of the first and/or second expressions of the embodiment of FIGS. 1-3, the medical instrument 10 includes a handpiece 54 attached to the proximal end 56 of the catheter 12, wherein the handpiece 54 includes the controller 50 with the controller 50 having a button 58 for a user to activate the controller 50. In one variation, as shown in FIG. 3, wires 60 connect the controller 50 to the windings 24, 26 and 28. In one modification of the example of the flexible accessory tube 246 having the accessory working channel 248, an end effector 262, such as a medical needle knife, is manually translatable within the accessory working channel 248 to extend from the accessory tube 246 for medical observation and/or medical treatment of the patient. Other types of medical end effectors include, without limitation, imagers, irrigators, cutting blades, wire snares, and ultrasound transducers.

A method of the invention is for using a medical instrument 10. The medical instrument 10 includes a flexible medical catheter 12, a catheter accessory device 14, and a linear motor controller 50. The catheter 12 has a distal end 16 insertable within a body lumen 18 of a patient 20. The device 14 slidably engages the catheter 12. One of the catheter 12 and the device 14 includes magnetic material 22, and the other of the catheter 12 and the device 14 includes electrically-energizable windings 24, 26 and 28. The catheter 12 and the device 14 are adapted to operate as a linear motor 30 wherein controllably electrically energizing the windings 24, 26 and 28 while immobilizing one of the catheter 12 and the device 14 slidingly moves the other of the catheter 12 and the device 14. The controller 50 is operatively connected to the windings 24, 26 and 28. The method includes inserting the distal end 16 of the catheter 12 within the body lumen 18. The method also includes immobilizing one of the catheter 12 and the device 14. The method also includes activating the controller 50 to advance the other of the catheter 12 and the device 14 within the body lumen 18.

In a first employment of the method, the device 14 is a flexible catheter-guiding member 36, wherein the catheter 12 includes a catheter lumen 38, wherein the member 36 is disposed in the catheter lumen 38, and wherein activating the controller 50 controllably electrically energizes the windings 24, 26 and 28 to slidingly move the catheter 12 along the member 36 when the member 36 is immobilized. In one extension, the method also includes manually advancing the member 36 slidingly through the catheter lumen 38 before immobilizing the member 36 and activating the controller 50.

In a second employment of the method, the catheter 212 includes an exterior rail 242, wherein activating the controller (the controller is shown as controller 50 in FIGS. 1 and 3) slidingly moves the device 214 along the exterior rail 242 when the catheter 212 is immobilized. In one extension, the method also includes manually advancing the catheter 212 before immobilizing the catheter 212 and activating the controller.

In one utilization of the method, the body lumen is a colon of a human or other mammal. In another utilization, the body lumen is an upper gastrointestinal tract. In a further utilization, the body lumen is an artery lumen. Other body lumens are left to those skilled in the art.

Several benefits and advantages are obtained from one or more of the expressions of an embodiment and the method of the invention. In a first example, the device is a medical guidewire which has been immobilized after having been manually advanced within the body lumen to extend from the catheter wherein controllably electrically energizing the windings slidingly moves the catheter along the guidewire. In one illustration of this example, the force to advance the catheter is applied proximate the distal end of the catheter so that the self-advancing catheter will not form undesirable loops, as can be appreciated by those skilled in the art. In a second example, the device includes a flexible accessory tube, and the catheter is immobilized after having been manually advanced within the body lumen wherein controllably electrically energizing the windings slidingly moves the accessory tube along an exterior rail of the catheter. In one illustration of this example, the force to advance the accessory tube is applied proximate the distal end of the accessory tube so that the self-advancing accessory tube will not form undesirable loops, as can be appreciated by those skilled in the art. In one employment of either or both examples, the self-advancing catheter and/or the self-advancing accessory tube has a greater advancement speed, than that obtained with manual advancement, which reduces the time required for a medical procedure.

While the present invention has been illustrated by a description of several expressions of embodiments and a method, it is not the intention of the applicants to restrict or limit the spirit and scope of the appended claims to such detail. Numerous other variations, changes, and substitutions will occur to those skilled in the art without departing from the scope of the invention. For instance, the medical instrument of the invention has application in robotic assisted surgery taking into account the obvious modifications of such systems, devices and methods to be compatible with such a robotic system. It will be understood that the foregoing description is provided by way of example, and that other modifications may occur to those skilled in the art without departing from the scope and spirit of the appended Claims.

What is claimed is:

1. A medical instrument comprising:
a) a flexible medical catheter having a distal end insertable within a body lumen of a patient; and
b) a catheter accessory device slidably engagable with the catheter, wherein one of the catheter and the device includes magnetic material, wherein the other of the catheter and the device includes electrically-energizable windings, and wherein the catheter and the device are adapted to operate as a linear motor wherein controllably electrically energizing the windings while immobilizing one of the catheter and the device slidingly moves the other of the catheter and the device.

2. The medical instrument of claim 1, wherein the catheter is a flexible endoscope insertion tube.

3. The medical instrument of claim 1, wherein the device is a flexible catheter-guiding member.

4. The medical instrument of claim 3, wherein the catheter includes a catheter lumen, and wherein the member is disposable in the catheter lumen.

5. The medical instrument of claim 4, wherein the catheter includes the windings, and wherein the windings are disposed proximate the distal end of the catheter.

6. The medical instrument of claim 5, wherein the member is chosen from the group consisting of a catheter guide wire and a catheter guide ribbon.

7. The medical instrument of claim 1, wherein the catheter includes an exterior rail, and wherein the device is adapted to slide along the exterior rail during operation of the linear motor.

8. The medical instrument of claim 7, wherein the device has a distal end, wherein the device includes the windings, and wherein the windings are disposed proximate the distal end of the device.

9. The medical instrument of claim 8, wherein the device includes a flexible accessory tube having an accessory working channel.

10. A medical instrument comprising:
a) a flexible medical catheter having a distal end insertable within a body lumen of a patient;
b) a catheter accessory device slidably engaging the catheter, wherein one of the catheter and the device includes magnetic material, wherein the other of the catheter and the device includes electrically-energizable windings, and wherein the catheter and the device are adapted to operate as a linear motor wherein controllably electrically energizing the windings while immobilizing one of the catheter and the device slidingly moves the other of the catheter and the device; and
c) a linear motor controller operatively connected to the windings.

11. The medical instrument of claim 9, wherein the catheter is a flexible endoscope insertion tube.

12. The medical instrument of claim 9, wherein the device is a flexible catheter-guiding member.

13. The medical instrument of claim 12, wherein the catheter includes a catheter lumen, wherein the member is disposed in the catheter lumen, and wherein the controller, when activated by a user, controllably electrically energizes the windings to slidingly move the catheter along the member when the member is immobilized by the user.

14. The medical instrument of claim 9, wherein the catheter includes an exterior rail, and wherein the device is adapted to slide along the exterior rail during activation of the controller.

15. The medical instrument of claim 14, wherein the device includes a flexible accessory tube having an accessory working channel and a distal accessory tube portion, and wherein the controller, when activated by a user, controllably electrically energizes the windings to slidingly move the distal accessory tube portion along the exterior rail when the catheter is immobilized by the user.

16. A method for using a medical instrument, wherein the medical instrument includes: a flexible medical catheter having a distal end insertable within a body lumen of a patient; a catheter accessory device slidably engaging the catheter, wherein one of the catheter and the device includes magnetic material, wherein the other of the catheter and the device includes electrically-energizable windings, and wherein the catheter and the device are adapted to operate as a linear motor wherein controllably electrically energizing the windings while immobilizing one of the catheter and the device slidingly moves the other of the catheter and the device; and a linear motor controller operatively connected to the windings, and wherein the method includes:
a) inserting the distal end of the catheter within the body lumen;
b) immobilizing one of the catheter and the device; and
c) activating the controller to advance the other of the catheter and the device within the body lumen.

17. The method of claim 16, wherein the device is a flexible catheter-guiding member, wherein the catheter includes a catheter lumen, wherein the member is disposed in the catheter lumen, and wherein activating the controller controllably electrically energizes the windings to slidingly move the catheter along the member when the member is immobilized.

18. The method of claim 17, also including manually advancing the member slidingly through the catheter lumen before immobilizing the member and activating the controller.

19. The method of claim 16, wherein the catheter includes an exterior rail, and wherein activating the controller slidingly moves the device along the exterior rail when the catheter is immobilized.

20. The method of claim 19, also including manually advancing the catheter before immobilizing the catheter and activating the controller.
